(19) United States
(12) Patent Application Publication
(10) Pub. No.: US 2026/0201468 A1
(43) Pub. Date: Jul. 16, 2026

(54) ARRAYS OF INTEGRATED ANALYTICAL DEVICES WITH IMPROVED COLLECTION EFFICIENCY AND USE OF THE ARRAYS IN OPTICAL ANALYSIS

(71) Applicant: Pacific Biosciences of California, Inc., Menlo Park, CA (US)

(72) Inventors: Hans CALLEBAUT, San Jose, CA (US); Arghavan ARJMAND, Santa Clara, CA (US); Mansoor A. SULTAN, Erbil (IQ); Jatadhari MISHRA, Mountain View, CA (US)

(73) Assignee: Pacific Biosciences of California, Inc., Menlo Park, CA (US)

(21) Appl. No.: 19/413,152

(22) Filed: Dec. 9, 2025

Related U.S. Application Data
(60) Provisional application No. 63/729,864, filed on Dec. 9, 2024.

(51) Int. Cl. C12Q 1/6874; G01N 21/64

(52) U.S. Cl. CPC C12Q 1/6874; G01N 21/6428; G01N 21/6452

(57) ABSTRACT

Arrays of integrated analytical devices and methods of optical analysis using the devices are provided. The arrays are useful in the analysis of highly multiplexed optical reactions in large numbers at high densities, including biochemical reactions, such as nucleic acid sequencing reactions. The devices allow the highly sensitive discrimination of optical signals using features such as spectra, amplitude, and time resolution, or combinations thereof. The devices include a nanophotonic patterned structure to increase the efficiency of detection of optical signals emitted from the optical reactions. Also provided are analytical systems containing an optical source and an array of integrated analytical devices.

### CROSS-REFERENCE TO RELATED APPLICATIONS

This application claims the benefit of U.S. Provisional Application No. 63/729,864, filed on Dec. 9, 2024, the disclosure of which is incorporated herein by reference in its entirety.

### BACKGROUND OF THE INVENTION

In analytical systems, the ability to increase the number of analyses being carried out at any given time by a given system has been a key component to increasing the utility and extending the lifespan of such systems. In particular, by increasing the multiplex factor of analyses with a given system, one can increase the overall throughput of the system, thereby increasing its usefulness while decreasing the costs associated with that use.

In optical analyses, increasing multiplex often poses increased difficulties, as it can require more complex optical systems, increased illumination or detection capabilities, and new reaction containment strategies. In some cases, systems seek to increase multiplex by many fold, and even orders of magnitude, which further implicate these considerations. Likewise, in certain cases, the analytical environment for which the systems are to be used is so highly sensitive that variations among different analyses in a given system may not be tolerable. These goals are often at odds with a brute force approach of simply making systems bigger and of higher power, as such steps often give rise to even greater consequences, e.g., in inter reaction cross-talk, decreased signal to noise ratios resulting from either or both of lower signal and higher noise, and the like. It would therefore be desirable to provide analytical systems that have substantially increased multiplex for their desired analysis, and particularly for use in highly sensitive reaction analyses, and in many cases, to do so while minimizing negative impacts of such increased multiplex.

At the same time, there is a continuing need to increase the performance of analytical systems and reduce the cost associated with manufacturing and using the system. In particular, there is a continuing need to increase the sensitivity and throughput of analytical systems, while at the same time reducing the size and complexity of analytical systems. There is a continuing need for analytical systems that have flexible configurations and that are easily scalable.

### SUMMARY OF THE INVENTION

In some aspects, the techniques described herein relate to an array of integrated analytical devices, at least one device including: a nanowell disposed in or through a surface layer of the at least one device; a nanoscale emission volume within the nanowell; a patterned structure positioned above the nanoscale emission volume; a detector layer positioned below the nanowell; and a sensing region positioned in the detector layer and optically coupled to the nanoscale emission volume; wherein a first portion of an optical signal emitted from the nanoscale emission volume is redirected by the patterned structure towards the sensing region.

In some aspects, the techniques described herein relate to an array, wherein the patterned structure is formed in a reflective surface of the at least one device.

In some aspects, the techniques described herein relate to an array, wherein the patterned structure is formed from a depression in a reflective surface of the at least one device.

In some aspects, the techniques described herein relate to an array, wherein the depression is between about 10 nm and about 100 nm deep.

In some aspects, the techniques described herein relate to an array, wherein the patterned structure is centered on the nanowell.

In some aspects, the techniques described herein relate to an array, wherein the reflective surface of the at least one device is a metallic surface.

In some aspects, the techniques described herein relate to an array, wherein the first portion of the optical signal redirected by the patterned structure forms a constructive interference with a second portion of the optical signal.

In some aspects, the techniques described herein relate to an array, wherein the patterned structure increases a signal-to-noise ratio in the at least one device compared to a device lacking the patterned structure.

In some aspects, the techniques described herein relate to an array, wherein the patterned structure decreases a cross-talk background signal in the at least one device compared to a device lacking the patterned structure.

In some aspects, the techniques described herein relate to an array, wherein the patterned structure includes a symmetric structure.

In some aspects, the techniques described herein relate to an array, wherein the patterned structure includes a ring structure, an interrupted ring structure, or a trench structure.

In some aspects, the techniques described herein relate to an array, wherein the ring structure or the interrupted ring structure has an inner radius of between about 0.05 μm and about 1 μm.

In some aspects, the techniques described herein relate to an array, wherein the ring structure or the interrupted ring structure has a width of between about 0.05 μm and about 1 μm.

In some aspects, the techniques described herein relate to an array, wherein the patterned structure has a depth of between about 0.01 μm and about 0.5 μm.

In some aspects, the techniques described herein relate to an array, wherein the surface layer includes a reflective surface, wherein the nanoscale emission volume is positioned on a bottom surface of the nanowell, wherein the optical signal emitted from the nanoscale emission volume displays a peak emission wavelength and is directed through a material having a refractive index, and wherein the reflective surface is spaced from the bottom surface of the nanowell at a vertical distance that is about an odd multiple of one fourth of the peak emission wavelength divided by the refractive index.

In some aspects, the techniques described herein relate to an array, wherein the patterned structure is formed in the reflective surface.

In some aspects, the techniques described herein relate to an array, wherein the vertical distance is about one fourth, about three fourths, or about five fourths of the peak emission wavelength divided by the refractive index.

In some aspects, the techniques described herein relate to an array, wherein the vertical distance is about three fourths of the peak emission wavelength divided by the refractive index.

In some aspects, the techniques described herein relate to an array, wherein the vertical distance is between about 100 nm and about 130 nm, between about 330 nm and about 360 nm, or between about 560 nm and about 590 nm.

In some aspects, the techniques described herein relate to an array, wherein the peak emission wavelength is between about 650 nm and about 700 nm.

In some aspects, the techniques described herein relate to an array, wherein the peak emission wavelength is about 670 nm.

In some aspects, the techniques described herein relate to an array, wherein the refractive index is between about 1.4 and about 1.5.

In some aspects, the techniques described herein relate to an array, wherein the at least one device further includes a laser rejection filter positioned between the nanowell and the detector layer and wherein the optical signal emitted from the nanoscale emission volume displays a peak emission wavelength that is less than about 100 nm above a 50% cutoff wavelength of the laser rejection filter.

In some aspects, the techniques described herein relate to an array, wherein the peak emission wavelength is from about 50 nm to about 100 nm above the 50% cutoff wavelength of the laser rejection filter.

In some aspects, the techniques described herein relate to an array, wherein the nanowell extends into an optically transparent layer below a top surface of the at least one device.

In some aspects, the techniques described herein relate to an array, wherein the patterned structure surrounds the nanowell within the optically transparent layer.

In some aspects, the techniques described herein relate to an array, wherein the optically transparent layer is an oxide layer.

In some aspects, the techniques described herein relate to an array, wherein a top surface of the at least one device includes an optically opaque layer.

In some aspects, the techniques described herein relate to an array, wherein the optically opaque layer is a metallic layer.

In some aspects, the techniques described herein relate to an array, wherein the patterned structure includes a dielectric material or a metal.

In some aspects, the techniques described herein relate to an array, wherein the at least one device further includes an optical excitation source positioned below the nanowell.

In some aspects, the techniques described herein relate to an array, wherein the optical excitation source delivers an optical illumination to the nanowell.

In some aspects, the techniques described herein relate to an array, wherein the patterned structure does not couple the optical illumination to the nanowell.

In some aspects, the techniques described herein relate to an array, wherein the optical excitation source includes an optical waveguide.

In some aspects, the techniques described herein relate to an array, wherein the at least one device further includes a laser rejection filter layer disposed between the optical waveguide and the detector layer.

In some aspects, the techniques described herein relate to an array, wherein the at least one device further includes a micromirror to direct an optical illumination from the optical excitation source to the nanowell.

In some aspects, the techniques described herein relate to an array, wherein the at least one device further includes at least one aperture layer disposed between the optical excitation source and the detector layer.

In some aspects, the techniques described herein relate to an array, wherein the at least one aperture layer includes titanium nitride.

In some aspects, the techniques described herein relate to an array, wherein the detector layer is included in a CMOS sensor.

In some aspects, the techniques described herein relate to an array, wherein the at least one device further includes a lens element disposed between the nanowell and the detector layer.

In some aspects, the techniques described herein relate to an array, wherein the lens element directs a portion of the optical signal from the nanoscale emission volume to the sensing region.

In some aspects, the techniques described herein relate to an array, wherein the at least one device further includes a color filtration layer disposed between the nanowell and the detector layer.

In some aspects, the techniques described herein relate to an array, wherein the at least one device includes an analyte disposed within the nanowell in fluidic contact with the nanoscale emission volume.

In some aspects, the techniques described herein relate to an array, wherein the analyte includes a biological sample.

In some aspects, the techniques described herein relate to an array, wherein the analyte includes a fluorophore.

In some aspects, the techniques described herein relate to an array, wherein the fluorophore is a FRET complex.

In some aspects, the techniques described herein relate to an array, wherein excitation of the FRET complex by an optical illumination source causes emission of a FRET signal that is optically coupled to the sensing region.

In some aspects, the techniques described herein relate to an array, wherein the analyte includes a sequencing mixture.

In some aspects, the techniques described herein relate to an array, wherein the sequencing mixture includes a fluorescent nucleotide that is bound by a DNA polymerase in a DNA polymerase-primer-template complex in the nanoscale emission volume.

In some aspects, the techniques described herein relate to an array, wherein the sequencing mixture includes a plurality of different fluorescent nucleotides that are bound by a DNA polymerase in a DNA polymerase-primer-template complex in the nanoscale emission volume, wherein the plurality of different fluorescent nucleotides are optically distinguishable by emission of a plurality of signal amplitudes at an excitation wavelength.

In some aspects, the techniques described herein relate to an array, wherein the array includes at least 1,000, at least 10,000, at least 100,000, at least 1,000,000, or at least 10,000,000 integrated analytical devices.

In some aspects, the techniques described herein relate to an array, wherein the nanoscale emission volume in the at least one device includes a DNA polymerase in a DNA polymerase-primer-template complex.

In some aspects, the techniques described herein relate to an array, wherein the nanowell in the at least one device includes a fluorescent nucleotide that is bound by the DNA polymerase in the DNA polymerase-primer-template complex in the nanoscale emission volume.

In some aspects, the techniques described herein relate to an array, wherein the nanowell in the at least one device includes a plurality of different fluorescent nucleotides that are bound by the DNA polymerase in the DNA polymerase-primer-template complex in the nanoscale emission volume, wherein the plurality of different fluorescent nucleotides are optically distinguishable by emission of a plurality of signal amplitudes at an excitation wavelength.

In some aspects, the techniques described herein relate to a method of optical analysis, including the step of detecting an optical signal from a target sample, wherein the optical signal is detected in an array of integrated analytical devices, wherein at least one device includes: a nanowell disposed in or through a surface layer of the at least one device; a nanoscale emission volume within the nanowell; a patterned structure positioned above the nanoscale emission volume; a detector layer positioned below the nanowell; and a sensing region positioned in the detector layer and optically coupled to the nanoscale emission volume; wherein an optical signal emitted from the nanoscale emission volume is detected at the sensing region; and wherein the patterned structure redirects a first portion of the optical signal from the nanoscale emission volume towards the sensing region.

In some aspects, the techniques described herein relate to a method, wherein the patterned structure is formed in a reflective surface of the at least one device.

In some aspects, the techniques described herein relate to a method, wherein the patterned structure is formed from a depression in a reflective surface of the at least one device.

In some aspects, the techniques described herein relate to a method, wherein the depression is between about 10 nm and about 100 nm deep.

In some aspects, the techniques described herein relate to a method, wherein the patterned structure is centered on the nanowell.

In some aspects, the techniques described herein relate to a method, wherein the reflective surface of the at least one device is a metallic surface.

In some aspects, the techniques described herein relate to a method, wherein the first portion of the optical signal redirected by the patterned structure forms a constructive interference with a second portion of the optical signal.

In some aspects, the techniques described herein relate to a method, wherein the patterned structure increases a signal-to-noise ratio in the at least one device compared to a device lacking the patterned structure.

In some aspects, the techniques described herein relate to a method, wherein the patterned structure decreases a cross-talk background signal in the at least one device compared to a device lacking the patterned structure.

In some aspects, the techniques described herein relate to a method, wherein the patterned structure includes a symmetric structure.

In some aspects, the techniques described herein relate to a method, wherein the patterned structure includes a ring structure, an interrupted ring structure, or a trench structure.

In some aspects, the techniques described herein relate to a method, wherein the ring structure or the interrupted ring structure has an inner radius of between about 0.05 μm and about 1 μm.

In some aspects, the techniques described herein relate to a method, wherein the ring structure or the interrupted ring structure has a width of between about 0.05 μm and about 1 μm.

In some aspects, the techniques described herein relate to a method, wherein the patterned structure has a depth of between about 0.01 μm and about 0.5 μm.

In some aspects, the techniques described herein relate to a method, wherein the surface layer of the at least one device includes a reflective surface, wherein the nanoscale emission volume is positioned on a bottom surface of the nanowell, wherein the optical signal emitted from the nanoscale emission volume displays a peak emission wavelength and is directed through a material having a refractive index, and wherein the reflective surface is spaced from the bottom surface of the nanowell at a vertical distance that is about an odd multiple of one fourth of the peak emission wavelength divided by the refractive index.

In some aspects, the techniques described herein relate to a method, wherein the patterned structure is formed in the reflective surface.

In some aspects, the techniques described herein relate to a method, wherein vertical distance is about one fourth, about three fourths, or about five fourths of the peak emission wavelength divided by the refractive index.

In some aspects, the techniques described herein relate to a method, wherein the vertical distance is about three fourths of the peak emission wavelength divided by the refractive index.

In some aspects, the techniques described herein relate to a method, wherein the vertical distance is between about 100 nm and about 130 nm, between about 330 nm and about 360 nm, or between about 560 nm and about 590 nm.

In some aspects, the techniques described herein relate to a method, wherein the peak emission wavelength is between about 650 nm and about 700 nm.

In some aspects, the techniques described herein relate to a method, wherein the peak emission wavelength is about 670 nm.

In some aspects, the techniques described herein relate to a method, wherein the refractive index is between about 1.4 and about 1.5.

In some aspects, the techniques described herein relate to a method, wherein the at least one device further includes a laser rejection filter positioned between the nanowell and the detector layer and wherein the optical signal emitted from the nanoscale emission volume displays a peak emission wavelength that is less than about 100 nm above a 50% cutoff wavelength of the laser rejection filter.

In some aspects, the techniques described herein relate to a method, wherein the peak emission wavelength is from about 50 nm to about 100 nm above the 50% cutoff wavelength of the laser rejection filter.

In some aspects, the techniques described herein relate to a method, wherein the nanowell extends into an optically transparent layer below a top surface of the at least one device.

In some aspects, the techniques described herein relate to a method, wherein the patterned structure surrounds the nanowell within the optically transparent layer.

In some aspects, the techniques described herein relate to a method, wherein the optically transparent layer is an oxide layer.

In some aspects, the techniques described herein relate to a method, wherein a top surface of the at least one device includes an optically opaque layer.

In some aspects, the techniques described herein relate to a method, wherein the optically opaque layer is a metallic layer.

In some aspects, the techniques described herein relate to a method, wherein the patterned structure includes a dielectric material or a metal.

In some aspects, the techniques described herein relate to a method, wherein the at least one device further includes an optical excitation source positioned below the nanowell.

In some aspects, the techniques described herein relate to a method, wherein the optical excitation source delivers an optical illumination to the nanowell.

In some aspects, the techniques described herein relate to a method, wherein the patterned structure does not couple the optical illumination to the nanowell.

In some aspects, the techniques described herein relate to a method, wherein the optical excitation source is an optical waveguide.

In some aspects, the techniques described herein relate to a method, wherein the at least one device further includes a laser rejection filter layer disposed between the optical waveguide and the detector layer.

In some aspects, the techniques described herein relate to a method, wherein the at least one device further includes a micromirror to direct an optical illumination from the optical excitation source to the nanowell.

In some aspects, the techniques described herein relate to a method, wherein the at least one device further includes at least one aperture layer disposed between the optical excitation source and the detector layer.

In some aspects, the techniques described herein relate to a method, wherein the at least one aperture layer includes titanium nitride.

In some aspects, the techniques described herein relate to a method, wherein the detector layer is included in a CMOS sensor.

In some aspects, the techniques described herein relate to a method, wherein the at least one device further includes a lens element disposed between the nanowell and the detector layer.

In some aspects, the techniques described herein relate to a method, wherein the lens element directs a portion of the optical signal from the nanoscale emission volume to the sensing region.

In some aspects, the techniques described herein relate to a method wherein the at least one device further includes a color filtration layer disposed between the nanowell and the detector layer.

In some aspects, the techniques described herein relate to a method, wherein the at least one device includes an analyte disposed within the nanowell in fluidic contact with the nanoscale emission volume.

In some aspects, the techniques described herein relate to a method, wherein the analyte includes a biological sample.

In some aspects, the techniques described herein relate to a method, wherein the analyte includes a fluorophore.

In some aspects, the techniques described herein relate to a method, wherein the fluorophore is a FRET complex.

In some aspects, the techniques described herein relate to a method, wherein excitation of the FRET complex by an optical illumination causes emission of a FRET signal that is optically coupled to the sensing region.

In some aspects, the techniques described herein relate to a method, wherein the analyte includes a sequencing mixture.

In some aspects, the techniques described herein relate to a method, wherein the sequencing mixture includes a fluorescent nucleotide that is bound by a DNA polymerase in a DNA polymerase-primer-template complex in the nanoscale emission volume.

In some aspects, the techniques described herein relate to a method, wherein the sequencing mixture includes a plurality of different fluorescent nucleotides that are bound by a DNA polymerase in a DNA polymerase-primer-template complex in the nanoscale emission volume, wherein the plurality of different fluorescent nucleotides are optically distinguishable by emission of a plurality of signal amplitudes at an excitation wavelength.

In some aspects, the techniques described herein relate to a method, wherein the at least one device is included in an array including at least 1,000, at least 10,000, at least 100,000, at least 1,000,000, or at least 10,000,000 integrated analytical devices.

In some aspects, the techniques described herein relate to a method, wherein the nanoscale emission volume in the at least one device includes a DNA polymerase in a DNA polymerase-primer-template complex.

In some aspects, the techniques described herein relate to a method, wherein the nanowell in the at least one device includes a fluorescent nucleotide that is bound by the DNA polymerase in the DNA polymerase-primer-template complex in the nanoscale emission volume.

In some aspects, the techniques described herein relate to a method, wherein the nanowell in the at least one device includes a plurality of different fluorescent nucleotides that are bound by the DNA polymerase in the DNA polymerase-primer-template complex in the nanoscale emission volume, wherein the plurality of different fluorescent nucleotides are optically distinguishable by emission of a plurality of signal amplitudes at an excitation wavelength.

In some aspects, the techniques described herein relate to a system for optical analysis including: a laser illumination source; and an array of integrated analytical devices optically coupled to the laser illumination source, at least one device including: a nanowell disposed in or through a surface layer of the at least one device; a nanoscale emission volume within the nanowell; a patterned structure positioned above the nanoscale emission volume; a detector layer positioned below the nanowell; and a sensing region positioned in the detector layer and optically coupled to the nanoscale emission volume; wherein a first portion of an optical signal emitted from the nanoscale emission volume is redirected by the patterned structure towards the sensing region.

In some aspects, the techniques described herein relate to a system, wherein the array includes at least 1,000, at least 10,000, at least 100,000, at least 1,000,000, or at least 10,000,000 integrated analytical devices.

In some aspects, the techniques described herein relate to a system, wherein the nanoscale emission volume in the at least one device includes a DNA polymerase in a DNA polymerase-primer-template complex.

In some aspects, the techniques described herein relate to a system, wherein the nanowell in the at least one device includes a fluorescent nucleotide that is bound by the DNA polymerase in the DNA polymerase-primer-template complex in the nanoscale emission volume.

In some aspects, the techniques described herein relate to a system, wherein the laser illumination source is optically coupled to the array of integrated analytical devices through a grating coupler.

In some aspects, the techniques described herein relate to a system, wherein the nanoscale emission volume in the at least one device is optically coupled to the grating coupler through an optical waveguide.

### BRIEF DESCRIPTION OF THE DRAWINGS

FIGS. 1A and 1B schematically illustrate an exemplary nucleic acid sequencing process that can be carried out using the disclosed arrays of integrated analytical devices.

FIG. 2 provides a schematic block diagram of an integrated analytical device.

FIG. 3A provides a partial schematic cross-sectional view of an exemplary unit cell of an integrated analytical device of the instant disclosure, where the device includes a patterned ring structure surrounding a nanowell. FIG. 3B shows a top-down scanning electron microscopic view of an array of nine partially-fabricated integrated analytical devices, where each partially-fabricated device includes an etched ring-shaped depression in the top waveguide cladding layer of the device. FIG. 3C shows a cross-sectional transmission electron microscopic side view of an integrated analytical device fabricated with features illustrated in FIG. 3A.

FIGS. 4A-4D show alternative ring design structures.

FIGS. 5A and 5B provide simulated results comparing the signal capture and crosstalk of exemplary integrated analytical device designs.

FIGS. 6A and 6B provide further simulated results comparing the signal capture and irradiance requirements of exemplary integrated analytical device designs.

FIGS. 7A-7C show an experimental comparison of various integrated analytical device designs.

### DETAILED DESCRIPTION OF THE INVENTION

### Integrated Analytical Devices

Multiplexed optical analytical systems are used in a wide variety of different applications. Such applications can include the analysis of single molecules, and can involve observing, for example, single biomolecules in real time as they carry out reactions. For ease of discussion, such multiplexed systems are discussed herein in terms of a preferred application: the analysis of nucleic acid sequence information, and particularly, single molecule nucleic acid sequence analysis. Although described in terms of a particular application, it should be appreciated that the applications for the devices and systems described herein are of broader application.

In the context of nucleic acid sequencing analyses, a single immobilized nucleic acid synthesis complex, comprising a polymerase enzyme, a template nucleic acid, whose sequence one is attempting to elucidate, and a primer sequence that is complementary to a portion of the template sequence, can be monitored in order to identify individual nucleotides as they are incorporated into the extended primer sequence. Incorporation is typically monitored by observing an optically detectable label on an added nucleotide, prior to, during, or following its incorporation. These single molecule primer extension reactions can be monitored in real-time, to identify the continued incorporation of nucleotides in the extension product and thus to elucidate the underlying template sequence. The process can also be referred to as single molecule real time (or SMRT®) sequencing.

Exemplary compositions and methods of forming a template for multipass sequencing, including complexing the template with a primer and a DNA polymerase, are described in U.S. Pat. No. 8,153,375, which is incorporated herein by reference in its entirety. Exemplary methods of real time nucleic acid sequencing using terminal-phosphate-labeled nucleotides are described in U.S. Pat. No. 7,361,466, which is incorporated herein by reference in its entirety. Exemplary Forster Resonance Energy Transfer (FRET) complexes suitable for providing the optical emission signals described herein, including FRET complexes that are distinguished by intensity but that have similar or the same emission spectra, are described in U.S. Pat. No. 8,927,212, which is incorporated herein by reference in its entirety. Exemplary methods of base calling using pulse widths and interpulse durations are described in U.S. Pat. No. 8,182,993, which is incorporated herein by reference in its entirety.

Exemplary analytical devices for monitoring optical reactions such as nucleic acid sequencing reactions are described in U.S. Patent Application Publication Nos. 2010/0065726, 2016/0061740, and 2023/0035224, which are incorporated herein by reference in their entireties. Such devices can be modified to include the patterned structures disclosed herein in order to improve the optical collection efficiencies of the devices. Analytical devices suitable for monitoring optical reactions are also described in U.S. Patent Application Publication No. 2014/0199016, and suitable aspects of those devices can also be combined with the patterned structures disclosed herein to improve the collection efficiencies of the devices. Any elements that would interfere with the effectiveness of the patterned structure in improving collection efficiency would, however, be omitted from the devices. For example, structural elements that increase the optical coupling of excitation light from an illumination waveguide to a nanowell, such as a ring structure positioned proximal to the bottom of the nanowell, may be omitted from the devices. As such, the devices may not have any reflective patterned structures that are closer to a nanoscale emission volume than the structures described herein.

Exemplary systems and methods for providing optical illumination to suitable arrays of analytical devices, such as providing laser illumination to the grating coupler of an array of analytical devices that is in turn coupled to one or more illumination waveguides for illuminating nanowells within the array, are described, for example, in U.S. Patent Application Publication No. 2016/0363728, which is incorporated herein by reference in its entirety.

Aspects of the disclosure include providing a sequencing mixture comprising a mixture of FRET complexes that are distinguished by the intensity of their optical emission but not by the peak wavelength of their optical emission spectra. Such FRET complexes can be coupled to nucleotides for use in nucleic acid sequencing reactions. The sequencing mixture can be in fluid contact with a nanoscale emission volume (see below), such that FRET complexes having different emission intensities occupy the nanoscale emission volume at different times, for example, when a FRET complex coupled to a nucleotide is bound by a DNA polymerase primer-template complex in the nanoscale emission volume. The devices and methods of sequencing described herein can accordingly comprise such sequencing mixtures, and the sequencing mixtures can include FRET complexes that are distinguished by the intensity of their optical emission but not by the peak wavelength of their optical emission spectra.

In preferred aspects, the immobilized template/polymerase primer complex is provided within an optically confined region, such as a zero mode waveguide (ZMW), or proximal to the surface of a transparent substrate, optical waveguide, or the like (see e.g., U.S. Pat. Nos. 6,917,726, and 7,170,50 and U.S. Patent Application Publication No. 2007/0134128, the full disclosure each of which is hereby incorporated by reference herein in its entirety for all purposes). The optically confined region is illuminated with an appropriate excitation radiation for the fluorescently labeled nucleotides that are to be used. Because the complex is within an optically confined region, or very small illumination volume, only the reaction volume immediately surrounding the complex is subjected to the excitation radiation. Accordingly, those fluorescently labeled nucleotides that are interacting with the complex, e.g., during an incorporation event, are present within the illumination volume for a sufficient time to identify them as having been incorporated. Although the analyte of interest in the devices disclosed herein is a template/polymerase primer complex that is incorporating fluorescently-labeled nucleotides, it should be understood that other analytes of interest, in particular fluorescent analytes of interest, can be monitored using the arrayed devices of the instant disclosure.

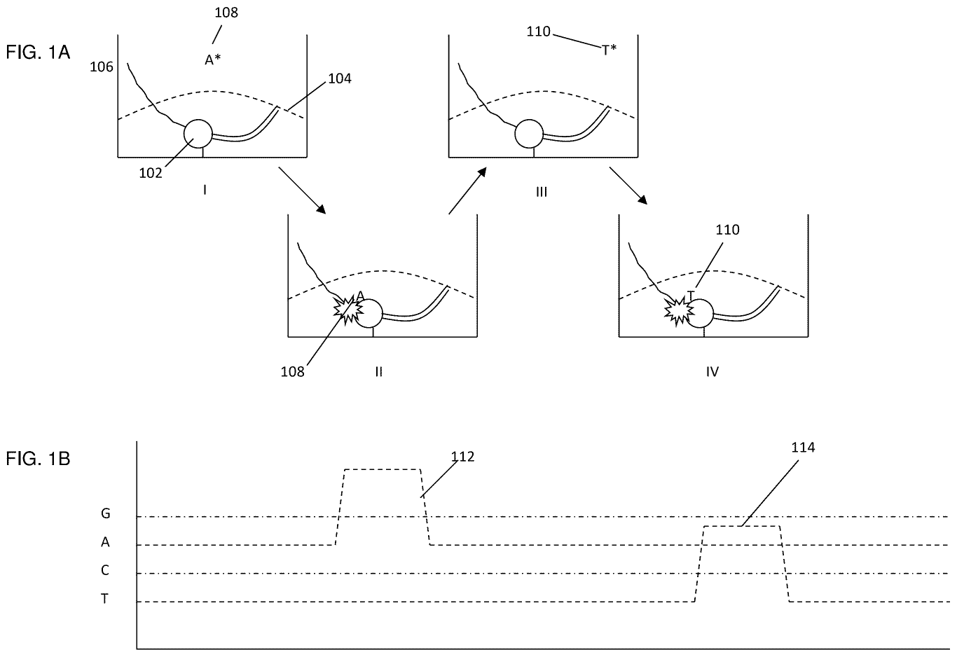

A schematic illustration of the just-described nucleic acid sequencing process is illustrated in FIGS. 1A and 1B. As shown in FIG. 1A, an immobilized complex 102 of a polymerase enzyme, a template nucleic acid and a primer sequence are provided within an observation volume (as shown by dashed line 104) of an optical confinement, of e.g., a zero mode waveguide 106. As an appropriate nucleotide analog, e.g., nucleotide 108, is incorporated into the nascent nucleic acid strand, it is illuminated for an extended period of time corresponding to the retention time of the labeled nucleotide analog within the observation volume during incorporation which produces a signal associated with that retention, e.g., signal pulse 112 as shown by the A trace in FIG. 1B. Once incorporated, the label that was attached to the polyphosphate component of the labeled nucleotide analog, is released. When the next appropriate nucleotide analog, e.g., nucleotide 110, is contacted with the complex, it too is incorporated, giving rise to a corresponding signal 114 in the T trace of FIG. 1B. By monitoring the incorporation of bases into the nascent strand, as dictated by the underlying complementarity of the template sequence, long stretches of sequence information of the template can be obtained.

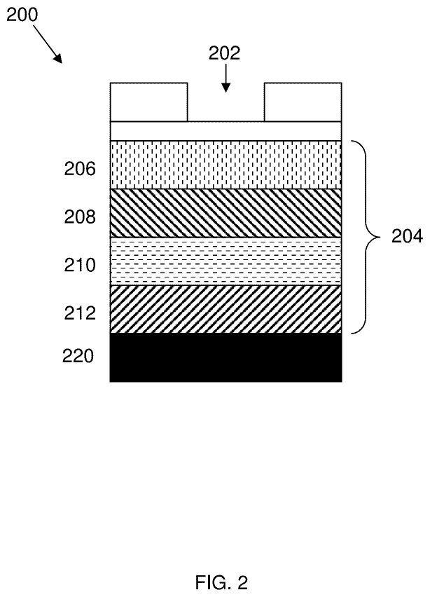

The above sequencing reaction can be incorporated into an array of devices, typically an array of integrated analytical devices, that provides for the simultaneous observation of multiple sequencing reactions, ideally in real time. While the components of each device and the configuration of the devices in the system can vary, each integrated analytical device typically comprises, at least in part, the general structure shown as a block diagram in FIG. 2. As shown, an integrated analytical device 200 typically includes a reaction cell 202, in which the analyte (i.e., the polymerase-template complex and associated fluorescent reactants) is disposed and from which the optical signals emanate. The analysis system further includes a detector element 220, which is disposed in optical communication with the reaction cell 202. Optical communication between the reaction cell 202 and the detector element 220 is provided by an optical train 204 comprised of one or more optical elements generally designated 206, 208, 210 and 212 for efficiently directing the signal from the reaction cell 202 to the detector 220. These optical elements generally comprise any number of elements, such as lenses, filters, gratings, mirrors, prisms, refractive material, apertures, or the like, or various combinations of these, depending upon the specifics of the application. By integrating these elements into a single device architecture, the efficiency of the optical coupling between the reaction cell and the detector is improved. Examples of integrated analytical systems, including various approaches for illuminating the reaction cell and detecting optical signals emitted from the reaction cell, are described in U.S. Patent Application Publication Nos. 2012/0014837, 2012/0019828, and 2012/0021525. Additional examples of integrated analytical systems, including systems comprising arrayed integrated analytical devices with highly efficient lens elements for the spatial separation and beam shaping of emission signals, are described in U.S. Patent Application Publication No. 2016/0061740. More specific examples of optical trains optimized for reduced-size integrated devices are described in U.S. Patent Application Publication No. 2023/0035224. Each of the above references is incorporated herein by reference in its entirety for all purposes.

As noted above, an analyte (e.g., a polymerase-template complex with associated fluorescent reactants) disposed within a reaction cell (e.g., element 202 in FIG. 2) or otherwise immobilized on the surface of the device, emits light that is transmitted to a detector element (e.g., element 220 in FIG. 2). For fluorescent analytes, the analyte is illuminated by an excitation light source, whereas for other analytes, such as chemiluminescent or other such analytes, an excitation light source may not be necessary. At least a portion of the reaction cell volume, the emission volume, is optically coupled to a sensing region positioned in the detector element, so that light emitted from an analyte within this volume, for example from a plurality of optical emitters within this volume, is measured by the sensing region. In order to maximize the number of analytes measured simultaneously, the size of the instant analytical devices is reduced as much as possible, so that the emission volume within each device is a nanoscale emission volume. Ideally, the optical coupling between the nanoscale emission volume and the detector element is highly efficient, in order to maximize the sensitivity of the device and maximize the signal output. Also important is the minimization of cross-talk between unit cells in an arrayed analytical system and the minimization of background noise caused by scattered or otherwise interfering optical energy from an excitation source, for example an excitation waveguide source.

Analytical reactions other than nucleic acid sequencing reactions can also be performed using the analytical devices and methods disclosed herein. For example, such reactions can include affinity binding reactions or enzymatic reactions. In the context of protein or peptide sequencing or profiling, binders to one or more epitopes of a subject protein or peptide can be labeled with a fluorophore (e.g., a FRET complex) that provides an optical emission signal. An epitope can be, for example, a short subsequence of amino acids, a single amino acid, or a post-translationally modified amino acid. In certain aspects, the real-time kinetics of interactions between one or more binders and one or more epitopes can be detected. Exemplary methods and compositions for protein sequencing, including the binding of N-terminal or C-terminal amino acids, are described in U.S. Patent Application Publication No. 2020/0209257.

Conventional analytical systems typically measure multiple spectrally distinct signals or signal events and must therefore utilize complex optical systems to separate and distinctly detect those different signal events. The optical path of an integrated device can be simplified, however, by a reduction in the amount or number of spectrally distinguishable signals that are detected. Such a reduction is ideally effected, however, without reducing the number of distinct reaction events that can be detected. For example, in an analytical system that distinguishes four different reactions based upon four different detectable signal events, where a typical system would assign a different signal spectrum to each different reaction, and thereby detect and distinguish each signal event, in an alternative approach, four different signal events would be represented by fewer than four different signal spectra, and would, instead, rely, at least in part, on other non-spectral distinctions between the signal events.

For example, a sequencing operation that would conventionally employ four spectrally distinguishable signals, e.g., a “four-color” sequencing system, in order to identify and characterize the incorporation of each of the four different nucleotides, could, in the context of an alternative configuration, employ a one-color or two-color analysis, e.g., relying upon a signals having only one or two distinct or distinguished spectral signals. However, in such an alternative configuration, this reduction in reliance on signal spectral complexity does not come at the expense of the ability to distinguish signals from multiple, i.e., a larger number of different signal producing reaction events. In particular, instead of relying solely on signal spectrum to distinguish reaction events, such an alternative configuration can rely upon one or more signal characteristics other than emission spectrum, including, for example, signal intensity, excitation spectrum, or both, to distinguish signal events from each other.

In one particular alternative configuration, the optical paths in an integrated analytical device can thus be simplified by utilizing signal intensity as a distinguishing feature between two or more signal events, such as between 4 signal events (e.g., one signal intensity corresponding to natural nucleobase). In its simplest iteration, and with reference to an exemplary sequencing process, two different nucleotides would bear fluorescent labels that each emit fluorescence under the same excitation illumination, i.e., having the same or substantially overlapping spectral band, and thus would provide benefits of being excited using a single excitation source. The resulting signals from each fluorescent label would have distinct signal intensities or amplitudes under that same illumination, and would therefore be distinguishable by their respective signal amplitudes. These two signals could have partially or entirely overlapping emission spectra, but separation of the signals based upon any difference in emission spectrum would be unnecessary. Even more usefully, four different nucleotides bearing fluorescent labels that each emit fluorescence under the same excitation illumination but having distinguishable emission intensities in response to that illumination can be distinguished by their respective signal amplitudes.

Accordingly, for analytical systems using two or more signal events that differ in signal amplitude, the integrated analytical devices of such systems can readily benefit through the removal of some or all of those components that would normally be used to separate spectrally distinct signals, such as multiple excitation sources and their associated optical trains, as well as the color separation optics, e.g., filters and dichroics, for the signal events, which in many cases, requires at least partially separate optical trains and detectors for each spectrally distinct signal. As a result, the optical paths for these integrated analytical devices are greatly simplified, allowing placement of detector elements in closer proximity to reaction cells, and improving overall performance of the detection process for these devices.

Provision of a signal-producing analyte that will produce different signal amplitudes under a particular excitation illumination profile can be accomplished in a number of ways. For example, different fluorescent labels can be used that present excitation spectral profiles that overlap but include different maxima. As such, excitation at a narrow wavelength will typically give rise to differing signal intensities for each fluorescent group. As will be appreciated, this same approach can be used with more than two label groups, where the resulting emission at a given excitation spectrum have distinguishable intensities or amplitudes.

Similarly, two different fluorescent labeling groups can have the same or substantially similar excitation spectra but provide different and distinguishable signal emission intensities due to the quantum yield of those labeling groups. Further, although described in terms of two distinct fluorescent dyes, it will be appreciated that each different labeling group can each include multiple labeling molecules. For example, each reactant can include an energy transfer dye pair that yields emissions of differing intensities upon excitation with a single illumination source. For example, a labeling group can include a donor fluorophore that is excited at a given excitation wavelength, and an acceptor fluorophore that is excited at the emission wavelength of the donor, resulting in energy transfer to the acceptor. By using different acceptors, whose excitation spectra overlap the emission spectrum of the donor to differing degrees, such an approach can produce overall labeling groups that emit at different signal amplitudes for a given excitation wavelength and level. Likewise, adjusting the energy transfer efficiency between the donor and acceptor will likewise result in differing signal intensities at a given excitation illumination.

Alternatively, different signal amplitudes can be provided by different multiples of signal producing label groups on a given reactant, e.g., putting a single label molecule on one reactant while putting 2, 3, 4, or more individual label molecules on a different reactant. The resulting emitted signal will be reflective of the number of labels present on a reactant and thus will be indicative of the identity of that reactant.

Exemplary compositions and methods relating to fluorescent reagents, such as nucleotide analogs, useful for the above purposes are described in, for example, U.S. Patent Application Publication Nos. 2009/0208957; 2010/0255488; 2012/0052506; 2012/0058469; 2012/0058473; 2012/0058482; 2012/0077189, 2013/0316912; 2015/0050659; 2016/0237279; 2017/0145495; 2017/0145496; and 2017/0145502; which is each incorporated by reference herein in its entirety for all purposes.

Accordingly, in preferred embodiments, the arrays of integrated analytical devices of the instant disclosure do not distinguish optical signals by color. In these embodiments, the devices therefore preferably do not include a color filtration element in their collection pathway, and each device preferably comprises a single sensing region, more specifically a single pixel, in a detector layer. Furthermore, in preferred embodiments, the integrated analytical devices of the instant arrays do not spatially separate an emission signal into more than one optical beam in the collection pathway.

As described above, integrated analytical devices making use of such approaches can see a reduction in complexity by elimination of spectral discrimination requirements, e.g., using signal amplitude or other non-spectral characteristics as a basis for signal discrimination. As shown in the block diagram of FIG. 2, an integrated analytical device 200 can include a reaction cell 202 that is defined upon or through a surface layer of the device. As shown in this drawing, the reaction cell comprises a nanowell disposed in or through the surface layer. Such nanowells can constitute depressions in a substrate surface or apertures disposed through one or more substrate layers to an underlying transparent substrate, e.g., as used in zero mode waveguide (ZMW) arrays (see, e.g., U.S. Pat. Nos. 7,181,122 and 7,907,800; see also below).

It should also be understood, however, that in some embodiments, the sample of interest can be confined in other ways, and that the nanoscale reaction cell in those embodiments can be omitted from the analytical devices. For example, if a target of interest is immobilized in a pattern on the surface of a device lacking separate reaction cells, binding events, or other events of interest, could be observed at those locations without the need for physical separation of the samples. Hybridization reactions, for example between immobilized nucleic acids and their complimentary sequences, or binding reactions, for example between antibodies and their ligands, where either member of the binding pair can be immobilized at a particular location on the surface of the device, could suitably be monitored using such an approach, as would be understood by those of ordinary skill in the art.

For samples requiring optical excitation, such as, for example, fluorescent samples, excitation illumination can be delivered to the nanowell, or to an otherwise immobilized target, from an excitation source that can either be separate from, or integrated into, the array of integrated analytical devices. For example, in the block diagram of FIG. 2, an optical waveguide (or waveguide layer) that is integrated into the device below the reaction cell 202 can be used to convey excitation light to the reaction cell or otherwise immobilized target, where an evanescent field emanating from the waveguide illuminates reactants within a portion of the sample volume known as the illumination volume. Use of optical waveguides to illuminate materials disposed upon or proximal to the surface of a substrate that includes a waveguide or waveguide array is described, for example, in U.S. Pat. No. 7,820,983, and in U.S. Patent Application Publication Nos. 2010/0065726 and 2014/0199016, the disclosures of which are incorporated herein by reference in their entireties for all purposes. The nanoscale reaction cell (also referred to herein as the “nanowell” or “ZMW”) can act to enhance the emission of fluorescence downward into the device and limit the amount of light scattered upwards. The emitted light, whether from a nanoscale reaction cell or from an immobilized target, is directed to the detector through an integrated optical train comprising one or more optical elements, as will be described in more detail below.

In some embodiments, excitation illumination can be delivered by an excitation source that is not necessarily integrated into the array of integrated analytical devices. For example, the reaction wells in the array can be simultaneously illuminated with a wide illumination source that covers a large number of reaction wells simultaneously, in what is termed “flood” illumination. Alternatively, an external excitation illumination beam can be scanned across the array of integrated devices to illuminate, in turn, each reaction well in the array. Such approaches have been described in detail in U.S. Pat. No. 8,247,216, which is incorporated herein by reference in its entirety. As is further described in U.S. Pat. No. 8,247,216, each reaction well in the array can be associated with a “micromirror” that is designed to improve both the optical excitation of a sample within the reaction well and the collection of optical emission signals from the device. Specifically, conical or parabolic mirrors can be integrated into a transparent substrate underlying the reaction well. The mirrors can be configured to redirect or to focus both the incoming and outgoing light to and from the reaction wells in an array of integrated analytical devices. In particular, the conical or parabolic mirrors can be fabricated from a reflective material, such as a metal layer, that is integrated into each device and that provides a reflective surface.

It should be understood in the context of the disclosure that the “optical coupling” of two components in a device is not intended to imply a directionality to the coupling. In other words, since the transmission of optical energy through an optical device is fully reversible, the optical coupling of a first component to a second component should be considered equivalent to the optical coupling of the second component to the first component.

Emitted signals from the reaction cell 202 that impinge on a sensing region of the detector layer 220 are then detected and recorded. The sensing region can correspond to a pixel or pixels in an array detector, for example a CMOS detector.

The detector layer is operably coupled to an appropriate circuitry, typically integrated into the substrate, for providing a signal response to a processor that is optionally included integrated within the same device structure or is separate from but electronically coupled to the detector layer and associated circuitry. Examples of types of circuitry are described in U.S. Patent Application Publication No. 2012/0019828.

As will be appreciated from the foregoing disclosure, the integrated analytical devices described above do not require the more complicated optical paths that are necessary in systems utilizing conventional four-color optics, obviating in some cases the need for excessive signal separation optics, dichroics, prisms, or filter layers. The scale of the devices can accordingly be reduced in order to accommodate an even higher level of multiplex in an array of such integrated devices.

Integrated Analytical Devices with Improved Collection Efficiency

As should be understood from the above, nanoscale integrated analytical devices, such as the above-described devices, are ideally designed to maximize the collection of optical signals emanating from an emission volume within a reaction well of a device, in order to maximize the sensitivity of the device. Any signal from the emission volume of a device that fails to reach the device's detector, for example any signal that is not directed towards the detector, either directly or by constructive redirection, is lost. Even worse, any signal not directed towards the detector can contribute to undesirable background crosstalk signals by being measured at the detector of a neighboring device.

The instant inventors have discovered that the inclusion of a nanophotonic structure in close proximity to the signal emitter (e.g., a dipole signal source) can increase the collection efficiency of the emitted light at the detector and can thus increase the signal-to-noise of each device. In some embodiments, the nanophotonic structure can be a ring-shaped aluminum depression in an oxide layer of the array, for example in a top waveguide cladding layer, surrounding the opening of the nanowell of each device, but other suitable patterned structures positioned around and above the emission volume can be designed to provide this function. In some embodiments, the nanophotonic structure is fabricated as part of an opaque metallic layer that covers the array of devices. Light emitted from the sample and reflected by the nanophotonic patterned structure above the emission volume can constructively interfere with light emitted from the sample in the direction of the detector layer below the emission volume and form a diffraction pattern. This diffraction pattern can be engineered according to well-known optical modelling methods to maximize the collection efficiency as measured by the ratio of detected power and emitted power. Of particular significance is the consideration that the increase in detected signal is achieved without placing any refractive or absorptive (i.e., lossy) material significantly closer to the optical excitation waveguide than is the case in existing designs, for example in any of the integrated analytical device designs described above.

Without intending to be bound by theory, it is believed that the just-described nanophotonic patterned structure enhances the detected signal by modulating the phase of the reflected light so that it interferes with the rest of the emission in a desired way (i.e. to maximize the signal power collected by the sensor). The structure provides additional dimensions to tailor the diffraction pattern beyond the current reflection off a flat surface. This allows for an enhanced collection efficiency and lower optical crosstalk at smaller pitch than would be possible without the structure. It should be understood that the main purpose of the nanophotonic structures is the modulation of the diffraction pattern of the dipole emitted light, and not an increase or modulation of the radiative modes available to the dipole (although this modulation can be unavoidable). An increase in density of radiative states can also be effected by the structure, which can be beneficial for lower quantum yield dyes.

The nanophotonic structures can be implemented, for example, by modulating the phase of the reflected light, e.g., by patterning an interposed layer of a different refractive index and leaving the high-reflectivity layer unpatterned. The high-reflectivity layer can be made, for example, out of alternating layers of dielectric material (distributed Bragg reflector or similar), or a patterning of said layers, although that may diminish the maximum possible collection efficiency. In preferred embodiments, however, the nanophotonic structure is patterned in an opaque metallic layer forming a top surface of the array of devices, for example on the surface of a top oxide layer cladding the optical excitation waveguide.

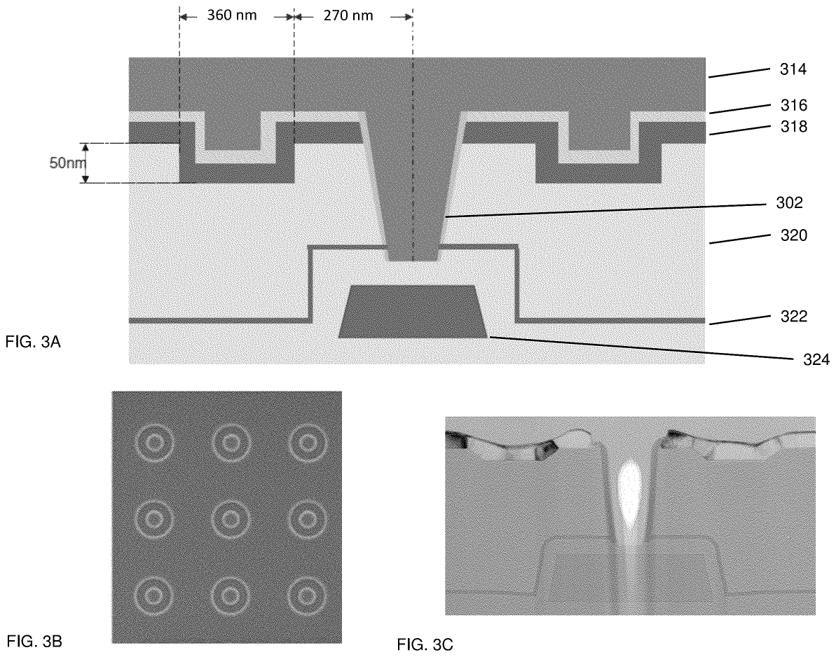

FIG. 3A shows a schematic cross-sectional view of a top portion of the unit cell of an exemplary integrated analytical device of the instant disclosure. The device includes a patterned ring structure fabricated in an upper layer of the device that is designed to improve the collection efficiency of an optical signal emanating from a nanowell in the device. Specifically, the drawing shows an aqueous sample solution 314 covering the device and filling nanowell 302. Also shown is an optional titanium nitride layer 316 (e.g., a TiN layer of approximately 20 nm thickness) on the surface of the device, a reflective layer 318 (e.g., an opaque metallic layer such as Al, of approximately 60 nm thickness) below the nitride layer, a waveguide cladding layer 320 (e.g., SiO₂) below the metallic layer, an etch stop layer 322 within the waveguide cladding, and an optical waveguide 324 (e.g., SiN of approximately 190 nm thickness) positioned to deliver optical excitation to the nanowell. In this example, the patterned ring structure is etched to a depth of approximately 50 nm in the waveguide cladding, so that deposition of the metallic layer during fabrication of the device creates the patterned ring structure. In this example, the inner edge of the ring structure has a diameter of approximately 270 nm, and the etched width of the ring structure is approximately 360 nm, but the dimensions can be modulated depending on the wavelength of optical emission and the materials used in fabrication of the device. The metallic layer of the device, which fills the etched portion of the waveguide cladding, reflects optical signal back toward the detector layer. It should also be understood that the metallic layer is preferably an opaque layer that is thick enough to block transmission of light from the aqueous sample solution above the device and thus to minimize background optical signals.

The detector layer in the device of FIG. 3A is not shown but would be positioned below the waveguide layer. Optional optical elements, such as lens elements or other types of light-directing or beam shaping elements, aperture layers, color filtration layers, laser rejection filter layers, and the like, can also be included between the nanowell and the detector layer, for example as described in U.S. Patent Application Publication Nos. 2016/0061740 and 2023/0035224, the disclosures of which are incorporated herein by reference in their entireties, and also as will be further described below. Such additional optical elements can, for example, spatially separate signal light, improve collection efficiency of signal light, reduce background signals, or serve other purposes. The optical waveguide could be omitted from the device if the optical excitation illumination is delivered to the nanowell by another source, for example by a flood illumination source.

FIG. 3B shows a top-down scanning electron microscopic (SEM) view of a partially fabricated array of nine devices, where the waveguide cladding layer (320) of FIG. 3A has been etched to form the patterned ring structures, but where the upper portions of the stack (i.e., the features above the waveguide cladding layer in FIG. 3A) have not yet been fabricated. FIG. 3C shows a transmission electron microscopic (TEM) side view of a single, fully-fabricated device having the structure essentially as shown schematically in FIG. 3A. The structure is designed to hold a sample with an emission signal positioned to emanate from the bottom of the nanowell, as indicated by the bright area in FIG. 3C.

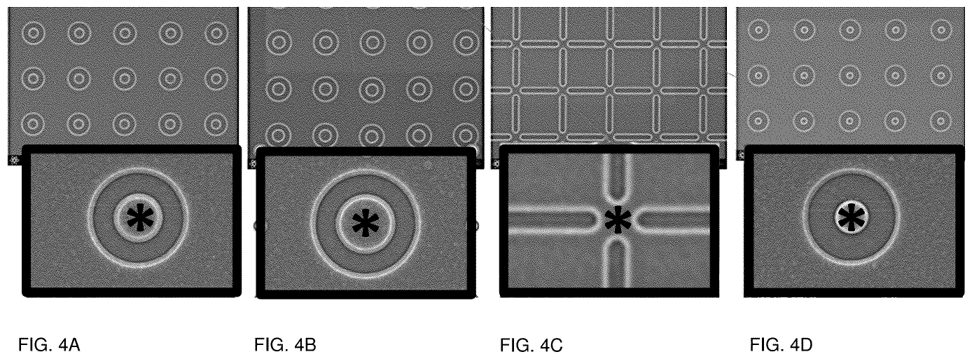

FIGS. 4A-4D show top-down SEM images of partially fabricated arrays of devices with alternative patterned structures fabricated in a top waveguide cladding layer of each device. In each case, the top image is an SEM image of a portion of the array, and the lower image is a magnified view of the top surface of a single device. FIGS. 4A, 4B, and 4D are ring-type patterned structures, where the rings have different radii or etched widths, and FIG. 4C is a cross-type or trench-type patterned structure. The location of the nanowell in each of the magnified views is indicated by an asterisk.

As just noted, the patterned structures can be designed and fabricated in a variety of shapes and dimensions, and the different device designs can be assessed for efficiency of signal capture and level of crosstalk both by simulations of modeled devices and by the fabrication and testing of device designs experimentally. In some embodiments, the patterned structure can be or can comprise a symmetric structure. For example, the patterned structure can be or can comprise a patterned ring structure, where the ring structure can either be a complete ring or can be interrupted by breaks around the ring structure. In some embodiments, the patterned structure can be or can comprise a trench structure, such as the trench structure illustrated in FIG. 4C.

In some embodiments, the ring structure or interrupted ring structure can have an inner radius of between about 0.1 μm and about 1 μm. For example, the structure can have an inner radius of about 100 nm, about 200 nm, about 300 nm, about 400 nm, about 500 nm, about 600 nm, about 700 nm, about 800 nm, about 900 nm, or even about 1 μm. In some embodiments, the ring structure or interrupted ring structure can have a width of between about 0.05 μm and about 1 μm. For example, the structure can have a width of about 50 nm, about 100 nm, about 200 nm, about 300 nm, about 400 nm, about 500 nm, about 600 nm, about 700 nm, about 800 nm, about 900 nm, or even about 1 μm.

In some embodiments, the patterned structure can have a depth of between about 0.01 μm and about 0.5 μm. For example, the structure can have a depth of about 10 nm, about 20 nm, about 30 nm, about 40 nm, about 50 nm, about 60 nm, about 70 nm, about 80 nm, about 90 nm, about 100 nm, about 150 nm, about 200 nm, about 300 nm, about 400 nm, or even about 0.5 μm. In some embodiments, the patterned structure can have a depth of about 0.5 μm, about 1 μm, about 2 μm, about 5 μm, or even deeper.

In some embodiments, the patterned structure can be positioned above the emission volume at a distance that is more than about 200 nm and less than about 1000 nm from the emission volume. In some embodiments, the patterned structure can be formed in a reflective layer or can be formed within 100 nm of a reflective layer. In some embodiments, the medium between emission volume and the patterned structure is not interrupted by any reflective structures.

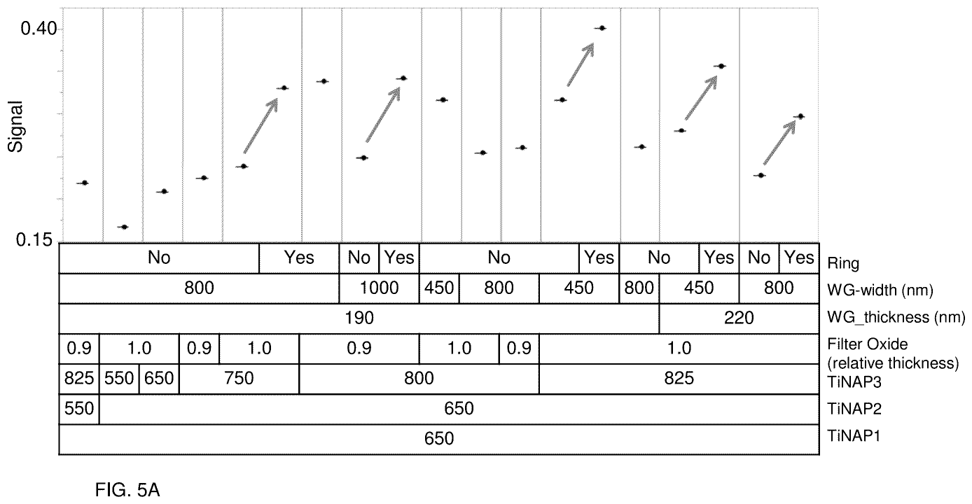

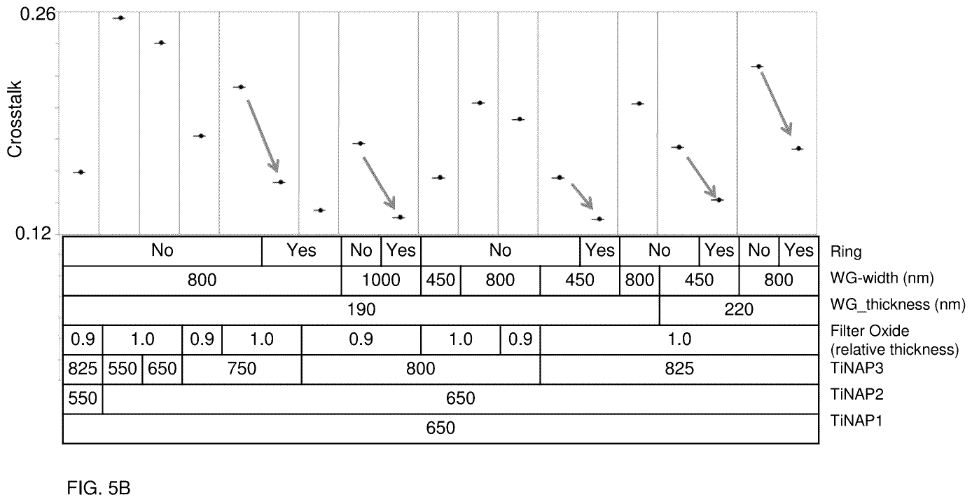

FIGS. 5A and 5B show the results of simulations with modeled integrated analytical devices having different designs and either including (“yes”) or not including (“no”) a patterned ring structure surrounding the nanowell (“Ring”). Also shown are other features of the devices, including waveguide width (“WG_width”) and thickness (“WG_thickness”) in nm, a scaling factor (0.9 or 1.0) for the relative thickness of the oxide layers in a laser rejection filter (“Filter Oxide (relative thickness)”), and the diameters of three titanium nitride aperture layers (“TiNAP1”, “TiNAP2”, and “TiNAP3”) in nm. Devices having these, or similar, features have been described, for example, in U.S. Patent Application Publication Nos. 2016/0061740 and 2023/0035224, the disclosures of which were previously incorporated by reference herein. As shown, devices including a patterned ring structure are predicted to display a roughly 30% boost in collected optical signal (“Signal”) (FIG. 5A) and a significant decrease in cross-talk between devices (“Xtalk”) (FIG. 5B). The simulations were run for patterned ring structures having an inner radius of 300 nm, a width of 200 nm, and a depth of 50 nm.

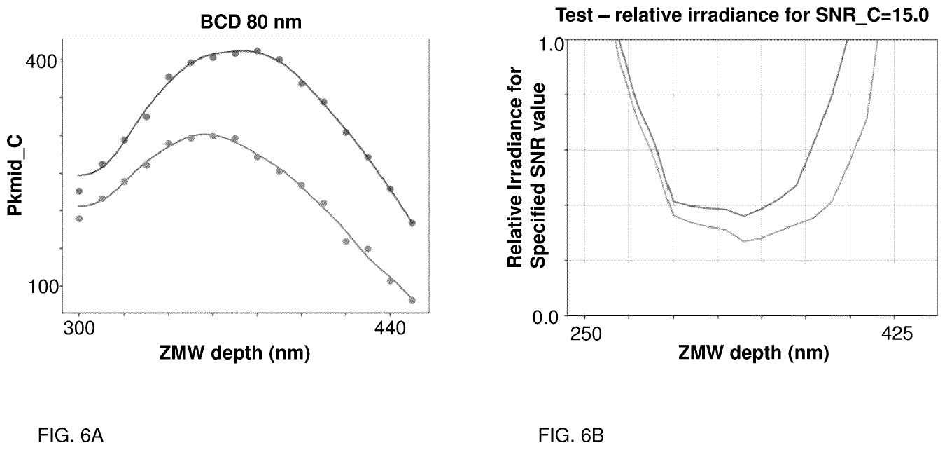

Integrated analytical devices having a patterned ring structure surrounding the nanowell and having different nanowell depths have also been shown in simulations to have an increased collection efficiency and improved signal-to-noise ratio (“SNR”) compared to devices lacking the patterned ring structure. Specifically, FIG. 6A shows a simulation of observed signal (“Pkmid”) as a function of nanowell depth (“ZMW depth”) for arrays of devices having a patterned ring structure (top curve; where the ring structure has an inner radius of 350 nm, a width of 200 nm, and a depth of 50 nm) and for arrays of devices lacking the patterned ring structure (bottom curve). These results predict a roughly 30% increase in collected signal for devices with the patterned ring structure. The nanowells in the modeled devices had a bottom diameter of approximately 80 nm.

The increased signal results in both an increase in accuracy of DNA sequencing and an increase in the yield of high fidelity results. Use of such devices can thus enable the use of lower-power optical sources for increased accuracy of results. For example, FIG. 6B shows a simulation that compares the required relative irradiance to achieve an arbitrary signal-to-noise ratio (“Relative irradiance for specified SNR value”) as a function of nanowell depth (“ZMW depth”) for arrays of devices having a patterned ring structure (bottom curve; where the ring structure has an inner radius of 350 nm, a width of 200 nm, and a depth of 50 nm) and for arrays of devices lacking this structure (top curve). These results demonstrate a decreased variability in performance within and between chips, a wider process window for optimal performance, and a reduced variability in laser power requirements for arrays of analytical devices comprising a patterned structure, and further in devices with optimized nanowell depths.

As demonstrated in the above simulations, the collection efficiencies in devices of the above designs can also depend significantly on the distance between the location of the signal emitter (typically positioned near the bottom of the nanowell) and the reflective surface (typically positioned at the top aperture of the nanowell), regardless of whether or not there is a patterned structure surrounding the nanowell above the emission volume. Without intending to be bound by theory, the proximity of the reflective surface to the emitter is understood to modulate the density of available emission modes to sharply enhance emission perpendicular to the reflective surface (and hence in the direction of the sensor) at specific distances. In particular, this effect is understood to be optimized when the distance between the emitter and the reflective surface approximates an odd multiple of the quarter wavelength of the emitted light in the medium between the emitter and the reflective surface divided by the refractive index of the medium. It should also be understood that the interruption in the reflector due to the nanowell aperture can result in a red shift of the peak collection wavelength that can depend on the size of the aperture.

The relationship between the above design features and the approximate optimal distance for collection efficiency can be represented according to the following equation:

$$Optimal distance \approx x * \frac{λ}{4 n}$$

where x is an odd integer, λ is the emission wavelength, and n is the refractive index.

The depth-dependent collection efficiency is illustrated in the simulated curves shown in FIG. 6A, where signal (“Pkmid_C”) is shown versus ZMW depth (i.e., the distance between the emitter at the bottom of the nanowell and the reflector surface for a peak emission wavelength of 670 nm in an SiO₂ medium (refractive index=1.46)). An optimal distance between the reflective surface and the nanoscale emission volume (e.g., the bottom of a nanowell) is therefore approximately one fourth of 670 nm divided by 1.46 (i.e., approximately 115 nm) or an odd multiple of this value (i.e., approximately 345 nm for 3×, approximately 575 nm for 5×, and so on). In other words, for values that are one fourth, three fourths, and five fourths of the peak emission wavelength divided by the refractive index, these distances may therefore be between about 100 and about 130 nm, between about 330 nm and about 360 nm, or between about 560 nm and about 590 nm. This optimal distance can correspond to the vertical depth of the nanowell.

A patterned structured, for example any of the patterned structures described herein, can be formed in the reflective surface and spaced as described above, or the patterned structure can be separate from the reflective surface.

In some embodiments, the optimal distance is within about 20% of the calculated value, within about 15% of the calculated value, within about 10% of the calculated value, within about 5% of the calculated value, or is even closer to the calculated value.

In some embodiments, the peak emission wavelength is between about 500 nm and about 850 nm, is between about 550 nm and about 800 nm, is between about 600 nm and about 750 nm, is between about 650 nm and about 700 nm, or is even about 670 nm.

In some embodiments, the refractive index of the medium between the emitter and the reflective surface is between about 1.3 and about 1.6, is between about 1.4 and about 1.5, or is even about 1.46.

In some embodiments, the at least one device further comprises a laser rejection filter positioned between the nanowell and the detector layer, for example any of the laser rejection filters described below. In these embodiments, the peak emission wavelength can be chosen, for example, to be less than about 200 nm, about 150 nm, or even about 100 nm above a 50% cutoff wavelength of the laser rejection filter. In some embodiments, the peak emission wavelength is chosen to be from about 50 nm to about 100 nm above the 50% cutoff wavelength of the laser rejection filter.

The design space for the patterned structures of the instant devices can be searched and optimized with the aid of models and simulations tools, such as a Finite Difference Time Domain (FDTD)-based code, for example as provided by Ansys Lumerical FDTD.

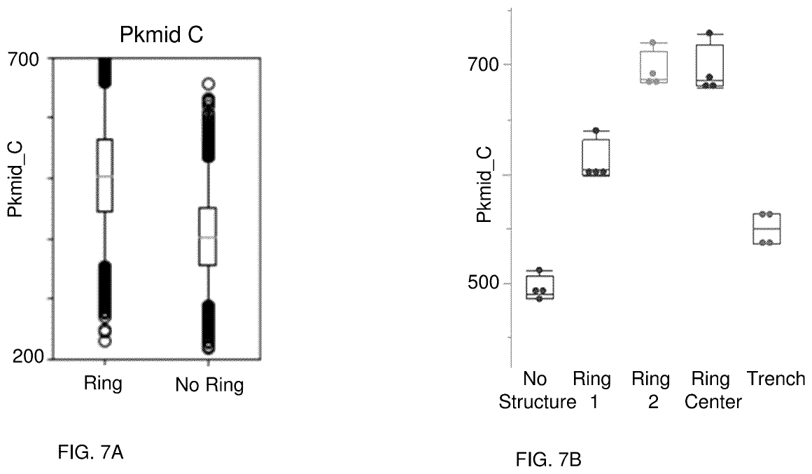

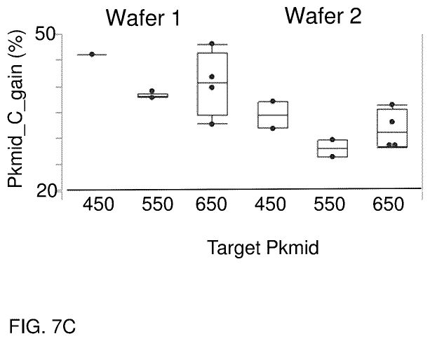

FIGS. 7A-7C show the experimental results of sequencing reactions performed on arrays of analytical devices fabricated with or without various exemplary patterned structures positioned above a nanoscale emission volume within a nanowell in each device. Measurements of the collected signal (“Pkmid_C”) for an optical stack with (“Ring”) or without (“No Ring”) a patterned ring structure are shown in FIG. 7A. Similar to the above-described simulations, these results demonstrate a roughly 25% increase in collected signal with devices that include a ring structure. The dependence of the signal enhancement on ring diameter in the patterned structure is explored in the results shown in FIG. 7B, where the observed signal for a basic optical stack with no patterned structure (“No Structure”) is compared with the signal for patterned rings of varying diameters: 250 nm (“Ring 1”), 300 nm (“Ring Center”), and 350 nm (“Ring 2”). All of the arrays of devices with patterned ring structures show higher signal than the arrays of devices with no patterned structure, with the 300 nm and 350 nm ring structures outperforming the 250 nm ring structure. Non-ring or partial-ring patterned structures (e.g. the trench structure referenced earlier) show moderate gains over devices lacking a patterned structure.

As described above, simulation results indicated that the optimal patterned ring width and consequent signal enhancement can depend on the detailed geometry of the signal collection path, most notably nanowell depth and exact lens dimensions. This variability was demonstrated experimentally, as illustrated in FIG. 7C, where the relative ratio of the signal increase to the basic signal (“Pkmid C_gain”) for measurements on two different wafers (“Wafer 1” and “Wafer 2”). The x-axis (target Pkmid) represents coupling of different excitation power levels (i.e., the number of photoelectrons detected within a frame). The y-axis shows the relative difference in signal collected in unit cells with and without the patterned structure.

It should also be understood that the nanophotonic patterned structures disclosed herein provide a different function than is provided by the local field enhancement elements disclosed, for example, in U.S. Patent Application Publication No. 2014/0199016. Those local field enhancement elements are designed and configured to increase the coupling efficiency of excitation illumination from an optical waveguide to a nanowell, for example by coupling energy from a waveguide core of high dielectric (such as Si₃N₄) through a cladding of low dielectric (such as SiO₂), rather than to increase the collection efficiency optical signals emanating from the emission volume. Furthermore, such a local field enhancement layer could potentially interfere with a patterned layer that is positioned above the emission volume and that directs emitted light toward the detector. Accordingly, in some embodiments, the arrayed devices of the instant disclosure do not comprise a local field enhancement layer, such as the local field enhancement layers disclosed in U.S. Patent Application Publication No. 2014/0199016 to increase illumination of a sample in a nanowell.

### Lens Elements

In some embodiments, the integrated analytical devices of the instant arrays further comprise at least one lens element disposed between the nanoscale emission volume and the detector layer. The at least one lens element can, in some embodiments, serve to focus and/or collimate emission light from the nanoscale emission volume. In other embodiments, the at least one lens element can serve to spatially separate the optical signal emitted from the nanoscale emission volume, for example as disclosed in U.S. Patent Application Publication No. 2016/0061740. More specifically, the at least one lens element can serve to direct light emitted from the nanoscale emission volume along two or more spatially separated optical paths at high efficiency. The spatial separation of emitted light preferably occurs prior color separation of the light, for example by a color filtration layer. In specific embodiments, the at least one lens element is a diffractive beam shaping element, such as those disclosed in U.S. Patent Application Publication No. 2016/0061740.

Improved Optical Collection Paths with Reduced Lateral Scale

In some embodiments, the integrated analytical devices of the instant arrays further comprise optical collection paths with reduced scale, for example unit cells with lateral dimensions of 2 μm or less. As noted in U.S. Patent Application Publication No. 2023/0035224, the disclosure of which was previously incorporated by reference herein, the collection efficiency of an optical stack can be increased while also maintaining a high signal-to-noise ratio even in integrated analytical devices with cropped lens elements, for example in devices with lateral dimensions less than 2 μm, through the use of novel light-gathering structures. Such light-gathering structures can be usefully combined with the features described herein to provide even higher collection efficiencies in devices with reduced lateral scale.

### Aperture Layers

As mentioned above, the integrated analytical devices of the instant disclosure can optionally include one or more aperture layers. The aperture layers are fabricated between or within other layers of the nanoscale analytical devices, for example between the ZMW/nanowell layer and an upper light-directing element layer, between an upper light-directing element layer and a lower light-directing element layer, between a lower light-directing element layer and a laser-rejection filter layer, and/or between a laser-rejection filter layer and a detector layer, for example as disclosed in U.S. Patent Application Publication No. 2023/0035224. The apertures provide openings to allow maximum transmission of emitted light from the ZMW/nanowell to the sensing regions of the detector element within a given unit cell, while at the same time minimizing background transmission of light, either from the excitation source (e.g., the waveguide), from autofluorescence within the device, or from cross-talk between adjacent unit cells. Aperture layers are typically constructed of light-blocking materials where transmission of light is undesirable and of transparent materials where transmission of light is desired. Suitable light-blocking materials for use in the aperture layers include, for example, titanium nitride, metals such as chromium, or any other appropriate light-blocking material. The light-blocking material is preferably titanium nitride. Suitable transparent materials for use in the aperture layers include, for example, SiO₂, Si₃N₄, Al₂O₃, TiO₂, GaP, and the like. In preferred embodiments, the aperture layer is approximately 100 nm thick.

### Laser Rejection Filter Elements

As described above, the integrated analytical devices of the instant disclosure can additionally and optionally include features designed to transmit certain wavelengths of light, while significantly decreasing or blocking other wavelengths of light. In particular, and as described above, it is desirable to transmit as much signal-related light as possible to the detector, and to block all, or at least most, noise-related light.

The arrayed integrated analytical devices of the instant disclosure can therefore additionally and optionally include one or more laser rejection filter elements within a laser rejection filter layer. The laser rejection filter layer is disposed between an excitation source and a detector layer of the integrated devices, typically between the nanowell and the detector layer and more specifically between an optical waveguide excitation source and the detector layer. In some embodiments, the laser rejection layer is disposed between a lens layer and/or a color filtration layer and the detector layer (e.g., as shown in FIG. 24 of U.S. Patent Application Publication No. 2016/0061740). In some embodiments, the laser rejection layer is disposed between a lower light-directing element and a detector layer (e.g., as shown in FIG. 4A of U.S. Patent Application Publication No. 2023/0035224) or between an upper light-directing element and a lower light-directing element (e.g., as shown in FIG. 4B of U.S. Patent Application Publication No. 2023/0035224). Such laser rejection filter elements (also known as pump rejection filters or scatter filters) are of particular importance in the case of fully integrated analytical devices, such as the devices of the instant disclosure, since the integrated nature of these devices can place constraints on the aggregate thickness of all layers, and can also increase the angular bandwidth over which the rejection must be assured. For a non-integrated detector device, the deposited layers responsible for rejection of non-signal light can be many tens of microns thick (summing over several filters participating), but typically only need to reject light over an angular range of <10 degrees (including both field of view (“FOV”) and filter tilt). For integrated devices such as the devices exemplified herein, however, the layers for pump rejection may need to be as thin as 5 microns or even less.

A further consideration with an integrated device is assuring that the rejected, non-signal light be terminated effectively (i.e., that it be efficiently removed from the optical system, for example by converting it to heat by absorption). For a non-integrated device, such termination is generally not critical, whereas for an integrated device, the reflected light can reach another detector site with a few (in principle, one) reflections, and furthermore, there is no local exit port for the rejected light to escape from the device. For these reasons, it is important to ensure that scattered light be converted to heat efficiently, ideally in one reflection. The detailed properties of two types of laser rejection filter elements suitable for use in the instant integrated devices is described in subsequent sections of the disclosure.

Suitable materials for use in the laser rejection filter elements of the instant devices include, for example, amorphous silicon/silicon oxide interference stacks, polymer-like resists, doped PECVD oxides, organo-silicone with absorbing dyes, and the like. In preferred embodiments, the laser rejection filter elements are thin-film interference filters. In more preferred embodiments, the laser rejection filter elements are prepared from layers of amorphous silicon and silicon oxide.

Similar laser-rejection filter designs have been described in U.S. Patent Application Publication No. 2016/0061740, previously incorporated herein by reference in its entirety.

### Multilayer and Hybrid Laser Rejection Filter Elements

An ideal laser rejection filter provides for the deep rejection of optical energy at the wavelengths of sample excitation (e.g., OD>=6 at 532 nm for a typical laser illumination source), displays a broad window of high transmission at the wavelengths of sample emission, and further displays a small Stokes shift between the wavelengths of interest. In addition, it is desirable for a laser rejection filter to display minimal dispersion with angle and polarization, minimal thickness, and controlled termination. Furthermore, the filter stacks are preferably inexpensive and readily manufacturable under conditions (e.g., temperatures) suitable for the manufacture of other components of an integrated device.

In the case of dielectric thin-film laser rejection filters, it can sometimes be challenging in the design of such stacks to obtain adequate filter performance over a wide range of incident angles for the non-signal light. For example, given a specified wavelength range, an edge filter can provide high reflection efficiency but only within a particular range of incident angles (typically from normal incidence up to a certain value). In some of the integrated device designs described herein, in order to keep the scattering photons of the excitation source from reaching the detector, rejection over a wide angular spectrum may be desirable, especially to block photons with higher angle of incidence than a typical thin film stack can adequately support.

This problem was addressed in U.S. Patent Application Publication No. 2016/0061740, which disclosed multilayer laser rejection filters comprising a low index total internal reflection (TIR) layer in order to reduce transmission of high angle scattering light. Specifically, the low index layer can be included in the device stack between the excitation source and the detector layer in order to minimize the background signal. Traditional dielectric long-pass filters reflect rays with lower angles of incidence (e.g., the middle rays in the drawing) more effectively than those with higher angle of incidence (e.g., the outer ray in the drawing). When this filter design is incorporated into an integrated device, the high angle scattering light from the waveguide has a relatively higher chance of being transmitted through the filter stack and reaching the sensor. As described in U.S. Patent Application Publication No. 2016/0061740, a low index TIR layer can be added between the integrated excitation waveguide and a low angle rejection filter, such as a dielectric filter stack. The high angle scattering light experiences total internal reflection upon encountering the low index TIR layer, and after multiple bounces, exits the integrated device from the side. At the same time, the lower angle scattering light is transmitted through the low index TIR layer but is rejected by the dielectric filter stack. The combined effect of the TIR layer and the filter stack thus results in a barrier filter that blocks the scattering light with wide angular spectrum.

One candidate material for the low index TIR layer of the multilayer filter stack is air, with almost zero dispersion and low refractive index, but other low index materials are also suitable, including other gases, liquids, and solids having low refractive index and other suitable properties. The specific choice of material for the low index TIR layer will depend on the refractive index and other physical properties of the adjacent layers, as would be understood by those of ordinary skill in the art.

To help collect the scattered light and reduce the chance of multiple scattering, an absorption layer or patch can optionally be added to the device. Materials for use in such an absorption layer are chosen based on their wavelength of absorption, their ability to dissipate optical energy, and their suitability in fabrication of the integrated device.

A variety of configurations of the above-described wide angular spectrum multilayer edge filter are possible, depending on the location, thickness, material choice, and number of layers of the low index layer(s). As described above, the low index layer can be placed directly below the excitation waveguide cladding, thus creating the shortest resonance cavity length and therefore limiting the chances for secondary scattering. The low index layer may, however, alternatively be placed within the thin film stack, or between the thin film stack and the detection layer. These configurations increase the resonance cavity length, and can therefore increase the chance of secondary scattering, but the configurations can advantageously facilitate manufacture of the device.

In any case, incorporation of an additional TIR design constraint into the laser rejection filter design generates added value to the low index layer. For example, by incorporating the low index layer (or layers) as an integral component in the laser rejection filter design, e.g., because the filter is no longer limited to the thin film stack but can include the layers from the excitation waveguide to the detection layer, the integrated device performance can be fully optimized.

The instant disclosure thus provides in another aspect arrays of integrated analytical devices with the above-described features for improving optical collection efficiency and further comprising optional laser rejection filter elements comprising a combination of dielectric stacks and absorption layers. Such hybrid filters take advantage of the complementary dependence on angle of incidence of interference coatings and absorption layers. Specifically, as mentioned above, interference coatings for rejection typically perform best for a cone centered on normal incidence, with dispersions that affect performance as a cosine of the angle in the interference thin films, whereas the performance of absorption rejection layers tends to increase with the angle of incidence, with dispersions that affect performance as a cosine of the angle in the absorbing layer. Owing to this complementary nature, a hybrid coating can be achieved with rejection of a target minimum over a wide angle range, in a minimum thickness. This thickness is reduced for higher refractive index thin films, and for lower refractive index absorbing layers. Note that thin films with absorption for the non-signal light (but minimal absorption of signal light) can be used effectively in a hybrid rejection filter.

As an example of an absorption dye suitable for use in combination with a dielectric filter stack, Aptina red1 has an absorption spectrum with high transmission above 600 nm. See Pang et al. (2011) Lab Chip 11:3698, FIG. 2. Although the thickness used in this publication was relatively large (8 μm), thinner layers can be used depending on the wavelength of laser excitation of the device. For example, a 5 μm layer provides OD>6 at 532 nm, a 4.7 μm layer provides OD>6 at 540 nm, and a 2.8 μm layer provides OD>6 at 562 nm. Other absorption dyes and pigments suitable for use in the instant hybrid filter stacks are readily identifiable by those of ordinary skill in the art.

In particular, laser rejection by an absorption dye layer, such as by a layer of Aptina red1 dye, advantageously displays no polarization dispersion, weak angle dispersion, and controlled termination of non-signal light. In addition, angularly non-uniform scatter can allow for further thinning of the absorption dye layer. If certain portions of the hemisphere have lower intensity non-signal light to be rejected, or if the intensity has known polarization dependence at some angles, this information can be used to further reduce the hybrid rejection filter thickness (for a given rejection target). The disadvantages of an absorption rejection filter, for example a layer of Aptina red1 dye, include a moderately large extinction coefficient, a relatively large thickness (5 μm), and the need to use sample dyes with a fairly large Stokes shift (532 nm to ~620 nm). These disadvantages can be offset to great extent, however, by the combination of an absorption layer with a dielectric stack in the instant hybrid rejection filters.

With respect to the dielectric stack component of a hybrid rejection filter, particularly advantageous rejection filters (especially those with low dependence on angle) are possible through the use of very high index materials for the interference portion of the filter. Exemplary materials finding utility for these purposes with 532 nm pumps are GaP (gallium phosphide) as the high index material, and TiO₂ as the low index material, although other suitable materials could be utilized, as described below, and as would be understood by those of ordinary skill in the art. Of note is that TiO₂ is typically used as a high index material for commonly produced coatings. The material also displays, however, a significant angular dispersion (with a blue shift) between 0 and 45 degrees, and a significant polarization dispersion (splitting) between a p-polarized optical signal (upper trace near 570 nm) and an s-polarized optical signal (middle trace near 570 nm).

The optical properties of the dielectric stack component of the hybrid rejection filter can be modulated as desired by the choice of materials used to construct the stack, by the thickness of each layer, and by the number of layers. The dielectric materials utilized to fabricate interference filters are generally nonconductive materials, typically metal salts and metal oxides, having a specific refractive index. Exemplary materials include SiO₂, SiO, Si₂O₃, Al₂O₃, BeO, MgO, CeF₃, LiF, NaF, MgF₂, CaF₂, TiO₂, Ta₂O₅, ZrO₂, HfO₂, Sb₂O₃, Y₂O₃, CeO₂, PbCl₂, and ZnS. Also of use is GaP, due to its extremely high refractive index. The dielectric stack is preferably designed with overall structure (H/2 L H/2)^{N}, where the H layer is a first material with relatively high refractive index and the L layer is a second material with relatively low refractive index. The physical thickness of each layer within the stack is chosen based on the desired optical properties, as is understood in the art. The value “N” is the number of repeating units of the structure within the parentheses and is an integer. Transmission in the stop band tends to zero (for a given incidence angle) with increasing overall thickness (e.g., as N increases).

It should be understood that the order of the coatings can be varied in order to achieve optimal performance of the hybrid laser rejection filter elements. For example, the layers can be ordered with absorption first, interference coatings second, or vice versa. The absorbing material can be carried in a host material such as PMMA, and can be shaped or patterned to fit within limited volumes or to permit simpler integration.

The coatings can be created in different process steps, and joined into an assembly, as would be understood by those of ordinary skill in the art.

In some embodiments, the laser rejection filter element is a multilayer or a hybrid rejection filter element.

In specific embodiments, the laser rejection filter element is a multilayer filter element comprising a dielectric interference filter layer and a low index total internal reflectance layer. In more specific embodiments, each of the devices further comprises an absorption layer.

In other specific embodiments, the laser rejection filter element is a hybrid rejection filter element comprising an absorption layer and a dielectric stack layer.

In some embodiments, the laser rejection filter element displays low optical transmission at 532 nm and high optical transmission above 620 nm.

Similar multilayer and hybrid laser rejection filters have been described in U.S. Patent Application Publication No. 2016/0061740, previously incorporated herein by reference in its entirety.

### Arrays of Integrated Analytical Devices

In order to obtain the volumes of sequence information that can be desired for the widespread application of genetic sequencing, e.g., in research and diagnostics, high throughput systems are desired. As noted above, and by way of example, in order to enhance the sequencing throughput of the system, multiple complexes are typically monitored, where each complex is sequencing a separate template sequence. In the case of genomic sequencing or sequencing of other large DNA components, these templates will typically comprise overlapping fragments of the genomic DNA. By sequencing each fragment, one can then assemble a contiguous sequence from the overlapping sequence data from the fragments.

As described above, and as shown in FIGS. 1A and 1B, the template/DNA polymerase-primer complex of such a sequencing system is provided, typically immobilized, within an optically confined region, such as a ZMW or nanowell, or proximal to the surface of a transparent substrate, optical waveguide, or the like. Preferably, such reaction cells are arrayed in large numbers upon a substrate in order to achieve the scale necessary for genomic or other large-scale DNA sequencing approaches. Such arrays preferably comprise a complete integrated analytical device, such as, for example, the device shown in the block diagram of FIG. 2 and in the unit cell partially illustrated in FIG. 3A.

Arrays of integrated analytical devices, such as arrays of devices comprising ZMWs/nanowells, can be fabricated at ultra-high density, providing anywhere from 1,000 ZMWs per cm², to 1,000,000 ZMWs per cm², or more. Thus, at any given time, it can be possible to analyze the reactions occurring in from 100, 1,000, 3,000, 5,000, 10,000, 20,000, 50,000, 100,000, 1 Million, 10 million, 25 million, 50 million, 100 million, or even more nanoscale emission volumes or other reaction regions within a single analytical system or even on a single substrate.

Using the foregoing systems, simultaneous targeted illumination of thousands or tens of thousands of ZMWs/nanowells in an array has been described. However, as the desire for multiplex increases, the density of ZMWs on an array, and the ability to provide targeted illumination of such arrays, increases in difficulty, as issues of ZMW cross-talk (signals from neighboring ZMWs contaminating each other as they exit the array), decreased signal:noise ratios arising from higher levels of denser illumination, and the like, increase. The arrays and methods of the instant disclosure address some of these issues.

### Methods of Optical Analysis

According to another aspect, the disclosure also provides methods of optical analysis using the arrays of integrated analytical devices disclosed herein. Advantageously, at least one device in the arrays used in these methods comprises a nanowell disposed in or through a surface layer of the at least one device, a nanoscale emission volume within the nanowell, a patterned structure positioned above the nanoscale emission volume, a detector layer positioned below the nanowell, and a sensing region positioned in the detector layer and optically coupled to the nanoscale emission volume. The methods of optical analysis include the step of detecting an optical signal from a target sample at the sensing region, wherein at least a first portion of the optical signal is redirected by the patterned structure towards the sensing region.

In some embodiments, the at least one device in the arrays used in these methods further comprises one or more of the above-described other specific device features, in any combination.

In some embodiments, the methods of optical analysis are directed to methods of nucleic acid sequencing. In these methods, the at least one device in the arrays used in these methods comprises a nucleic acid template/polymerase primer complex immobilized within an optically confined region in the nanowell and a plurality of fluorescently labeled nucleotides in fluid contact with the nucleic acid template/polymerase primer complex. In these methods, the optically confined region is illuminated with an excitation wavelength, a fluorescent signal from the optically confined region is measured at the sensing region, and at least one of plurality of fluorescently labeled nucleotides is identified by the measured fluorescent signal.

In specific method embodiments, the array used in the method comprises at least 100 integrated analytical devices.

In other specific method embodiments, the array used in the method has a density of at least 1000 integrated devices per cm².

In still other specific method embodiments, the array used in the method is associated with an excitation light source. More specifically, the excitation light source is coupled to the array through an optical coupler integrated in the array.

### Integrated Analytical Systems for Optical Analysis

According to another aspect, the disclosure provides improved integrated analytical systems for optical analysis. These systems comprise an optical source, such as a laser illumination source, and any of the arrays of integrated analytical devices disclosed herein. In particular, the arrays of integrated analytical devices included in these systems can comprise a nanowell disposed in or through a surface layer of the at least one device; a nanoscale emission volume within the nanowell; a patterned structure positioned above the nanoscale emission volume; a detector layer positioned below the nanowell; and a sensing region positioned in the detector layer and optically coupled to the nanoscale emission volume, wherein a first portion of an optical signal emitted from the nanoscale emission volume is redirected by the patterned structure towards the sensing region.

As should be understood from the above disclosure, these systems can provide improved optical collection efficiencies by maximizing the collection of optical signals emanating from an emission volume within a reaction well of each device and thus maximizing the sensitivity of each device, and by minimizing cross-talk between devices within the array.

In some embodiments, the array of integrated analytical devices in these systems can comprise at least 1,000, at least 10,000, at least 100,000, at least 1,000,000, or even at least 10,000,000 integrated analytical devices.

More specifically, the nanoscale emission volume in the at least one device of the array can comprise a DNA polymerase in a DNA polymerase-primer-template complex.

Even more specifically, the nanowell in the at least one device can comprise a fluorescent nucleotide that is bound by the DNA polymerase in the DNA polymerase-primer-template complex in the nanoscale emission volume.

In some embodiments, the laser illumination source in these systems is optically coupled to the array of integrated analytical devices through a grating coupler. In specific embodiments, the nanoscale emission volume in the at least one device is optically coupled to the grating coupler through an optical waveguide.

It will be readily apparent to one of ordinary skill in the relevant arts that other suitable modifications and adaptations to the methods and applications described herein can be made without departing from the scope of the invention or any embodiment thereof.

All patents, patent publications, and other published references mentioned herein are hereby incorporated by reference in their entireties as if each had been individually and specifically incorporated by reference herein.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined by reference to the appended claims, along with their full scope of equivalents.

1. An array of integrated analytical devices, at least one device comprising:
a nanowell disposed in or through a surface layer of the at least one device;
a nanoscale emission volume within the nanowell;
a patterned structure positioned above the nanoscale emission volume;
a detector layer positioned below the nanowell; and
a sensing region positioned in the detector layer and optically coupled to the nanoscale emission volume;
wherein a first portion of an optical signal emitted from the nanoscale emission volume is redirected by the patterned structure towards the sensing region.

2. The array of claim 1, wherein the patterned structure is formed in a reflective surface of the at least one device.

3. The array of claim 1, wherein the patterned structure is formed from a depression in a reflective surface of the at least one device.

4-6. (canceled)

7. The array of claim 1, wherein the first portion of the optical signal redirected by the patterned structure forms a constructive interference with a second portion of the optical signal.

8. The array of claim 1, wherein the patterned structure increases a signal-to-noise ratio in the at least one device compared to a device lacking the patterned structure.

9. The array of claim 1, wherein the patterned structure decreases a cross-talk background signal in the at least one device compared to a device lacking the patterned structure.

10. The array of claim 1, wherein the patterned structure comprises a symmetric structure.

11. The array of claim 1, wherein the patterned structure comprises a ring structure, an interrupted ring structure, or a trench structure.

12-13. (canceled)

14. The array of claim 1, wherein the patterned structure has a depth of between about 0.01 μm and about 0.5 μm.

15. The array of claim 1, wherein the surface layer comprises a reflective surface, wherein the nanoscale emission volume is positioned on a bottom surface of the nanowell, wherein the optical signal emitted from the nanoscale emission volume displays a peak emission wavelength and is directed through a material having a refractive index, and wherein the reflective surface is spaced from the bottom surface of the nanowell at a vertical distance that is about an odd multiple of one fourth of the peak emission wavelength divided by the refractive index.

16-22. (canceled)

23. The array of claim 1, wherein the at least one device further comprises a laser rejection filter positioned between the nanowell and the detector layer and wherein the optical signal emitted from the nanoscale emission volume displays a peak emission wavelength that is less than about 100 nm above a 50% cutoff wavelength of the laser rejection filter.

24. (canceled)

25. The array of claim 1, wherein the nanowell extends into an optically transparent layer below a top surface of the at least one device.

26-27. (canceled)

28. The array of claim 1, wherein a top surface of the at least one device comprises an optically opaque layer.

29. (canceled)

30. The array of claim 1, wherein the patterned structure comprises a dielectric material or a metal.

31. The array of claim 1, wherein the at least one device further comprises an optical excitation source positioned below the nanowell.

32. The array of claim 31, wherein the optical excitation source delivers an optical illumination to the nanowell.

33-35. (canceled)

36. The array of claim 31, wherein the at least one device further comprises a micromirror to direct an optical illumination from the optical excitation source to the nanowell.

37. The array of claim 31, wherein the at least one device further comprises at least one aperture layer disposed between the optical excitation source and the detector layer.

38. (canceled)

39. The array of claim 1, wherein the detector layer is comprised in a CMOS sensor.

40. The array of claim 1, wherein the at least one device further comprises a lens element disposed between the nanowell and the detector layer.

41. (canceled)

42. The array of claim 1, wherein the at least one device further comprises a color filtration layer disposed between the nanowell and the detector layer.

43. The array of claim 1, wherein the at least one device comprises an analyte disposed within the nanowell in fluidic contact with the nanoscale emission volume.

44. The array of claim 43, wherein the analyte comprises a biological sample.

45. The array of claim 43, wherein the analyte comprises a fluorophore.

46-47. (canceled)

48. The array of claim 43, wherein the analyte comprises a sequencing mixture.

49-50. (canceled)

51. The array of claim 1, wherein the array comprises at least 1,000, at least 10,000, at least 100,000, at least 1,000,000, or at least 10,000,000 integrated analytical devices.

52. The array of claim 51, wherein the nanoscale emission volume in the at least one device comprises a DNA polymerase in a DNA polymerase-primer-template complex.

53-114. (canceled)
